(19) United States
(12) Patent Application Publication
(10) Pub. No.: US 2023/0173242 A1
(43) Pub. Date: Jun. 8, 2023

(54) DEVICE FOR SUBDERMAL INSERTION OF SOLID MEDIA

(71) Applicant: Eastern Virginia Medical School, Norfolk, VA (US)

(72) Inventors: Meredith Roberts Clark, Norfolk, VA (US); Andrea Thurman, Norfolk, VA (US); Gustavo Doncel, Norfolk, VA (US); M. Melissa Peet, Norfolk, VA (US); Jason Robinson, Cambridge, MA (US); Oivind Brockmeier, Medford, MA (US)

(21) Appl. No.: 18/073,799

(22) Filed: Dec. 2, 2022

Related U.S. Application Data
(60) Provisional application No. 63/285,294, filed on Dec. 2, 2021.

(51) Int. Cl. A61M 37/00

(52) U.S. Cl. CPC A61M 37/0069; A61M 2205/583; A61M 2205/586

(57) ABSTRACT

Various implementations include a device for subdermal insertion of solid media. The device includes a handle having a central plane, a needle, and a guide. The needle is coupled to and extending from the handle. The needle has a needle longitudinal axis and a distal tip disposed along the needle longitudinal axis and the central plane. The guide extends from the handle. The guide has a distal end disposed along the central plane. The distal end of the guide is disposed further than the distal tip of the needle from the handle. The distal tip of the needle is visible when viewed along the central plane and perpendicular to an axis including the distal end of the guide and the distal tip of the needle.

### BACKGROUND

Current subcutaneous insertion devices for drug-eluting implants have the advantage of being reusable and simple but lack important needle safety features that may prevent accidental needle sticks and reuse without proper cleaning and sterilization.

Further, the depth of implant insertion in many current insertion devices is not well controlled, leading to implant migration or improper placement. To account for this, some current subdermal insertion devices include depth guides. However, these depth guides are located directly above the needle during insertion, blocking the user’s view of the tip of the needle during use.

Furthermore, many of the current devices are not preloaded and require loading of the solid media into the trocar needle by the user. Because the needles are not preloaded under sterile conditions, these devices create a risk of contamination and infection. The current devices that are preloaded with solid media are only able to be used for the preloaded drug specific to that implant and, therefore, lack versatility.

Thus, a need exists for an implant insertion device that can be used safely with minimal training, does not obstruct the view of the needle during use, and can be used with a variety of solid media and implant forms.

### SUMMARY

Various implementations include a device for subdermal insertion of solid media. The device includes a handle, a needle, and a guide. The handle has a central plane. The needle is coupled to and extends from the handle. The needle has a needle longitudinal axis and a distal tip disposed along the needle longitudinal axis and the central plane. The guide extends from the handle. The guide has a distal end disposed along the central plane. The distal end of the guide is disposed further than the distal tip of the needle from the handle. The distal tip of the needle is visible when viewed along the central plane and perpendicular to an axis including the distal end of the guide and the distal tip of the needle.

In some implementations, an axis including the distal end of the guide and the distal tip of the needle forms an entry angle with the needle longitudinal axis. In some implementations, the entry angle is in the range of 5-50 degrees. In some implementations, the entry angle is in the range of 5-30 degrees. In some implementations, the entry angle is in the range of 5-15 degrees.

In some implementations, the guide defines a window through which the distal tip of the needle is visible.

In some implementations, the handle defines a cavity and the needle is retractable into the handle. In some implementations, the needle is manually retractable. In some implementations, the needle is retractable by a spring force. In some implementations, the handle includes a slider for causing the needle to move from an extended position to a retracted position. In some implementations, the device further includes a plunger axially aligned with the needle longitudinal axis such that, when the needle is retracted, the plunger extends through the needle. In some implementations, the needle is lockable in a retracted position. In some implementations, the needle is lockable in an extended position.

In some implementations, the handle has an hourglass shape as viewed in a direction parallel to the central plane.

In some implementations, an edge of the distal end of the guide closest to the distal tip of the needle defines a fillet, a chamfer, or an eased edge.

In some implementations, the needle includes a first needle coupling portion and the handle includes a second needle coupling portion and the first needle coupling portion of the needle is coupled to the second needle coupling portion of the handle. In some implementations, the needle and the handle include rotational clocking features to ensure the rotational position of the needle relative to the handle. In some implementations, the first needle coupling portion is integrally formed with the needle. In some implementations, the needle is preloaded with one or more pellets, rods, capsules, cylinders, spheres, implants, sensors, or any combination thereof.

In some implementations, the guide includes at least one surface that defines a plane that extends parallel to the needle longitudinal axis. In some implementations, a total width of the at least one surface is 15 mm-30 mm.

In some implementations, the guide includes at least one surface that defines a plane that extends parallel to the needle longitudinal axis. In some implementations, a total width of the at least one surface is three or more times larger than an outer diameter of the needle.

In some implementations, the guide includes a surface that is closest to the needle. In some implementations, a distance from the surface of the guide to the needle is 3 mm-15 mm. In some implementations, the distance from the surface of the guide to the needle is 5 mm-10 mm.

Various other implementations include a device for subdermal insertion of solid media. The device includes a handle and a guide. The handle has a central plane and a second needle coupling portion for coupling a first needle coupling portion of a needle. The needle has a needle longitudinal axis and a distal tip disposed along the needle longitudinal axis. When the needle is coupled to the needle coupling portion, the needle extends from the handle such that the distal tip of the needle is disposed along the central plane. The guide extends from the handle. The guide has a distal end disposed along the central plane.

In some implementations, the handle includes a rotational clocking feature to ensure the rotational position of the needle relative to the handle.

In some implementations, the handle has an hourglass shape as viewed in a direction parallel to the central plane.

In some implementations, an axis including the distal end of the guide and the distal tip of the needle forms an entry angle with the needle longitudinal axis when the needle is coupled to the needle coupling portion of the handle. In some implementations, the entry angle is in the range of 5-50 degrees. In some implementations, the entry angle is in the range of 5-30 degrees. In some implementations, the entry angle is in the range of 5-15 degrees.

In some implementations, the handle defines a cavity and the needle is retractable into the handle when the needle is coupled to the needle coupling portion of the handle. In some implementations, the needle is manually retractable. In some implementations, the needle is retractable by a spring force. In some implementations, the handle includes a slider for causing the needle to move from an extended position to a retracted position. In some implementations, the handle includes a plunger axially aligned with the needle longitudinal axis such that, when the needle is retracted, the plunger extends through the needle. In some implementations, the needle is lockable in a retracted position. In some implementations, the needle is lockable in an extended position.

In some implementations, when the needle is coupled to the needle coupling portion of the handle, an edge of the distal end of the guide closest to the distal tip of the needle defines a fillet, a chamfer, or an eased edge.

In some implementations, the needle is preloaded with one or more pellets, rods, capsules, cylinders, spheres, implants, sensors, or any combination thereof.

In some implementations, when the needle is coupled to the needle coupling portion of the handle, the distal end of the guide is disposed further than the distal tip of the needle from the handle, and the distal tip of the needle is visible when viewed along the central plane and perpendicular to an axis including the distal end of the guide and the distal tip of the needle. In some implementations, the guide defines a window through which the distal tip of the needle is visible.

In some implementations, the guide includes at least one surface that defines a plane that extends parallel to the needle longitudinal axis when the needle is coupled to the needle coupling portion of the handle. In some implementations, a total width of the at least one surface is 15 mm-35 mm.

In some implementations, the guide includes at least one surface that defines a plane that extends parallel to the needle longitudinal axis. In some implementations, a total width of the at least one surface is three or more times larger than an outer diameter of the needle.

In some implementations, the guide includes a surface that is closest to the needle when the needle is coupled to the needle coupling portion of the handle. In some implementations, a distance from the surface of the guide to the needle is 3 mm-15 mm. In some implementations, the distance from the surface of the guide to the needle is 5 mm-10 mm.

### BRIEF DESCRIPTION OF DRAWINGS

Example features and implementations are disclosed in the accompanying drawings. However, the present disclosure is not limited to the precise arrangements and instrumentalities shown.

FIG. 1 is a perspective view of a device for subdermal insertion of solid media, according to one implementation.

FIGS. 2A and 2B are perspective views of the device of FIG. 1 with the needle uncoupled from the handle and the needle coupled to the handle, respectively.

FIGS. 3A and 3B are left side views of the device of FIG. 1 with the needle uncoupled from the handle and the needle coupled to the handle, respectively.

FIG. 3C is a right side view of the device of FIG. 1 with the needle coupled to the handle.

FIGS. 4A and 4B are top and bottom views of the device of FIG. 1 with the needle coupled to the handle.

FIGS. 5A and 5B are partially exploded perspective views of the device of FIG. 1 in the extended position and the retracted position, respectively.

FIGS. 6A-6D are cross-sectional side views of coupling a needle to a device, according to another implementation, and retracting the needle of the device.

FIG. 7 is a perspective view of various needle and solid media sizes that are usable with the device of FIG. 1-5B.

FIG. 8 is a top view of a device, according to another implementation.

FIG. 9 is a perspective view of a device, according to another implementation, and a cassette.

FIG. 10 is a perspective view of a needle, according to another implementation.

FIG. 11 is a perspective view of the device of FIG. 8.

FIG. 12 is a side view of a device for subdermal insertion of solid media, according to another implementation.

FIG. 13 is a top view of the device of FIG. 12.

FIG. 14 is a perspective view of the device of FIG. 12.

FIG. 15 is another perspective view of the device of FIG. 12.

FIG. 16 is a side view of a device for subdermal insertion of solid media, according to another implementation.

FIG. 17 is a top view of the device of FIG. 16 with a needle coupled to the handle.

FIG. 18 is a top view of the needle of FIG. 17 without the device.

FIG. 19 is a rear perspective view of the device of FIG. 16 with a needle coupled to the handle.

FIG. 20 is a rear perspective view of the device of FIG. 19 with the needle uncoupled from the handle.

FIG. 21 is a front perspective view of the device of FIG. 16 with a needle coupled to the handle.

FIG. 22 is a front perspective view of the device of FIG. 21 with the needle uncoupled from the handle.

### DETAILED DESCRIPTION

The devices, systems, and methods disclosed herein provide for subdermal insertion of solid media. The devices disclosed herein can be used to insert bio-degradable or non-biodegradable solid drug dosage forms, in particular drug pellets or rod-like implants, under the skin of a human or animal, comprising a design that is particularly easy and quick to use, ergonomic, safe, and suitable for use by minimally-trained, entry-level and lay providers in busy clinical settings.

Some implementations of the devices include a guide for controlling the depth and angle of insertion of the needle into a patient. The guide includes a window or other structure to allow a user to view the distal tip of the needle from above the guide as the needle is being inserted into the patient. Some implementations of the devices also include a needle coupling portion to which a needle can be coupled. This allows for a generic device that is capable of being coupled to needles of different sizes and diameters that are preloaded with a desired solid media.

Various implementations include a device for subdermal insertion of solid media. The device includes a handle having a central plane, a needle, and a guide. The needle is coupled to and extending from the handle. The needle has a needle longitudinal axis and a distal tip disposed along the needle longitudinal axis and the central plane. The guide extends from the handle. The guide has a distal end disposed along the central plane. The distal end of the guide is disposed further than the distal tip of the needle from the handle. The distal tip of the needle is visible when viewed along the central plane and perpendicular to an axis including the distal end of the guide and the distal tip of the needle.

Various other implementations include a device for subdermal insertion of solid media. The device includes a handle and a guide. The handle has a central plane and a needle coupling portion for coupling a needle. The needle has a needle longitudinal axis and a distal tip disposed along the needle longitudinal axis. When the needle is coupled to the needle coupling portion, the needle extends from the handle such that the distal tip of the needle is disposed along the central plane. The guide extends from the handle and has a distal end disposed along the central plane.

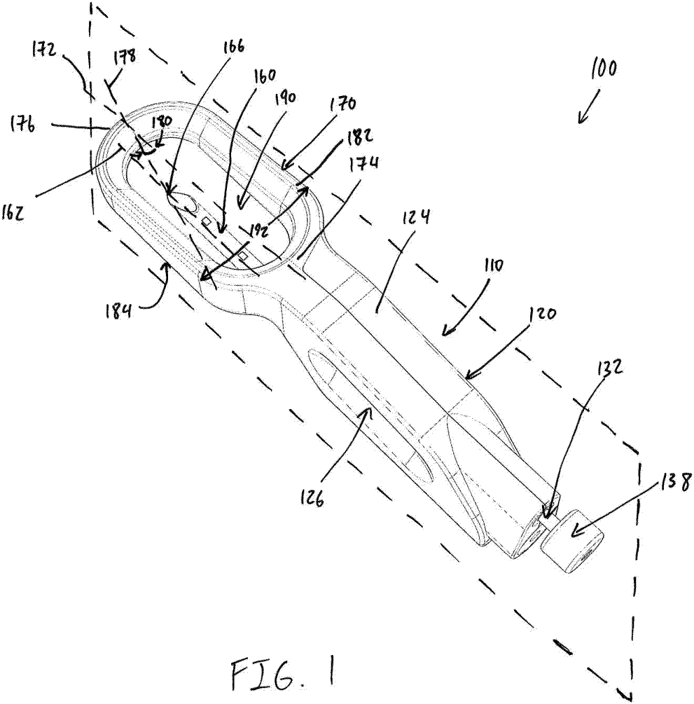

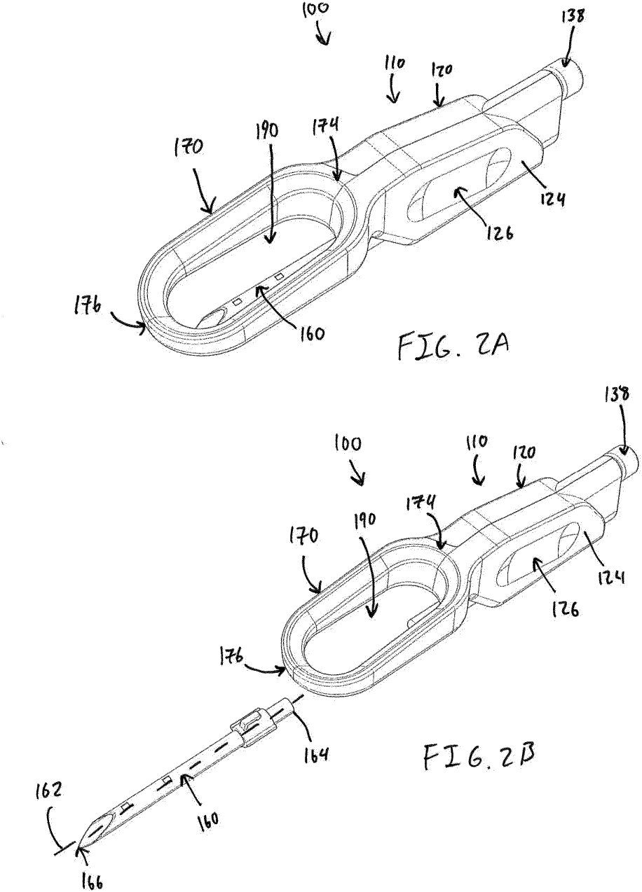

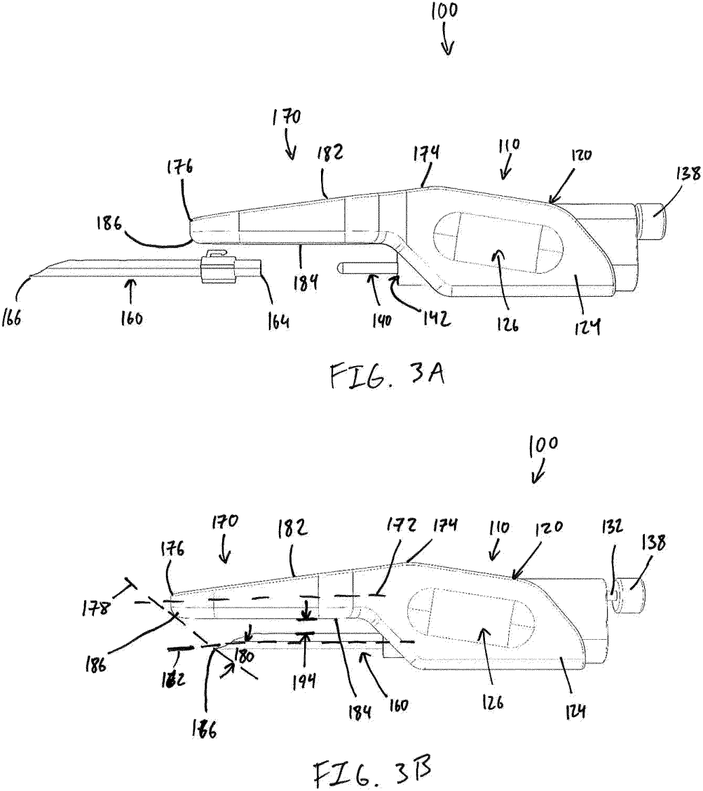

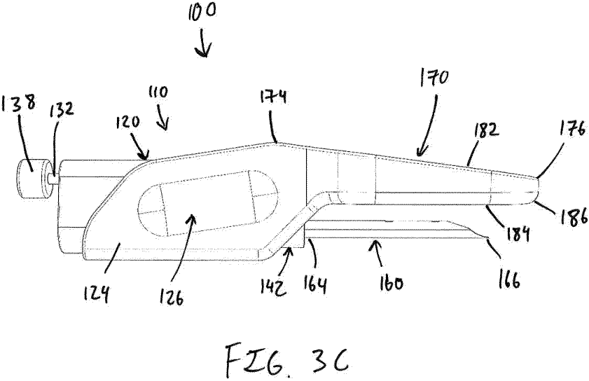

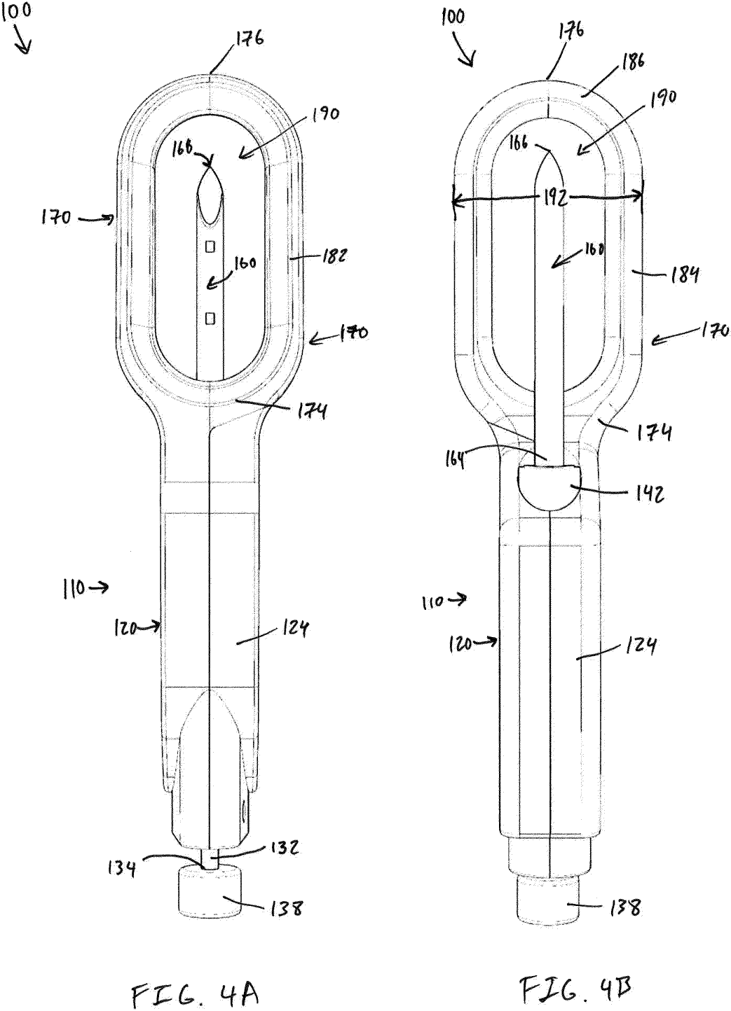

FIG. 1-5B show a device 100 for subdermal insertion of solid media, according to one implementation. The device 100 includes a handle 110, a needle 160, and a guide 170.

The handle 110 has a central plane 112 and includes a handle body 120 defining cavity 122. An outer surface 124 of the handle body 120 defines two concave grip portions 126 located on opposite sides of the central plane 112. A user can position a portion of the hand of the user within the grip portions 126 during use of the device 100 to prevent hand slipping.

Although the grip portions 126 shown in FIG. 1-5B are concave features, in other implementations, the grip portions are convex, flat, or any combination of concave, convex, and/or flat features. In some implementations, the grip portions include a textured surface or a friction coating or material.

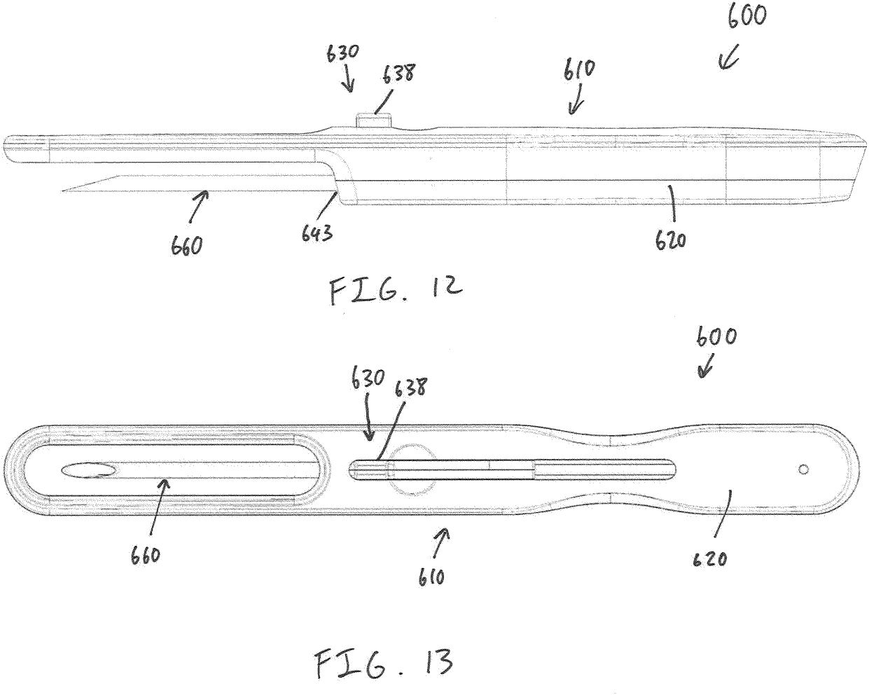

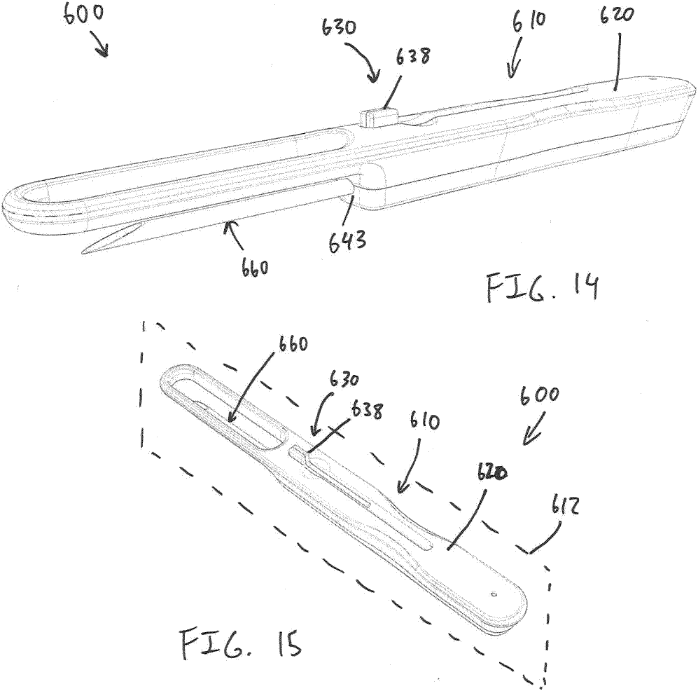

In some implementations, like the implementation of the device 600 shown in FIGS. 12-15, the handle 610 body 620 can have an hourglass-shaped portion as viewed from the top of the handle 610 in a direction parallel to the central plane 612. The hourglass shape of the handle 612 body 620 provides an affordance to push and pull against during insertion and retraction of the needle 660.

As seen in FIG. 1-5B, the handle 110 includes a retractor 130 disposed within the cavity 122 of the handle body 120. The retractor 130 includes a pull rod 132 that is slidable along its longitudinal axis 139 relative to the handle body 120, a plunger 140 that is disposed parallel to the longitudinal axis 139 of the pull rod 132 and static relative to the handle body 120, and a needle coupling portion 142, 143. The needle coupling portion includes a first needle coupling portion 142 that is part of the needle 160 and a second needle coupling portion 143 that is a part of the retractor 130 of the handle 110. The plunger 140 and the second needle coupling portion 143 of the retractor 130 are disposed along the central plane 112 of the handle 110 such that, when the first needle coupling portion 142 of a needle 160 is coupled to the second needle coupling portion 143 of the retractor 130, the needle 160 is also disposed along the central plane 112. The second needle coupling portion 143 defines an opening 144, and the plunger 140 is disposed within the opening 144 of the second needle coupling portion 143 such that the second needle coupling portion 143 is slidable along the plunger 140. The pull rod 132 has a first end 134 and a second end 136 opposite the first end 134. The first end 134 of the pull rod 132 includes a pull knob 138, and the second end 136 of the pull rod 132 is coupled to the second needle coupling portion 143.

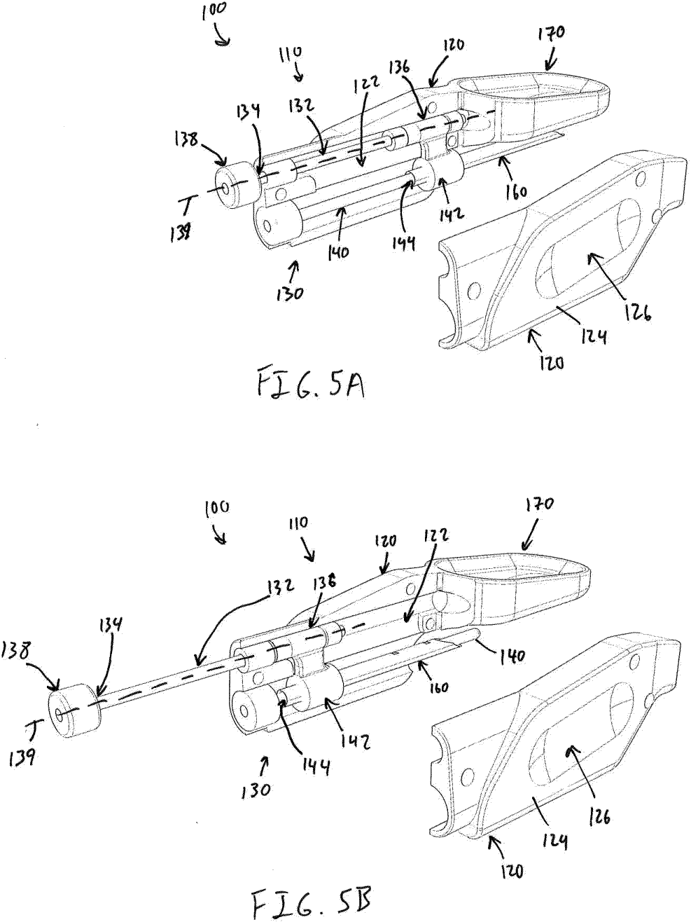

When a user exerts a force on the first end 134 of the pull rod 132 in a direction away from the handle body 120, the pull rod 132 is extended out of the handle body 120. Because the second needle coupling portion 143 is coupled to the second end 136 of the pull rod 132, the second needle coupling portion 143 slides along the plunger 140 in the same direction as the pull rod 132. When the first needle coupling portion 142 of a needle 160 is coupled to the second needle coupling portion 143, the movement of the second needle coupling portion 143 causes the needle 160 to move from an extended position shown in FIG. 5A in which at least a portion of the needle 160 extends outside of the cavity 122 of the handle body 120 to a retracted position shown in FIG. 5B in which the needle 160 is disposed within the cavity 122 of the handle body 120, as discussed below.

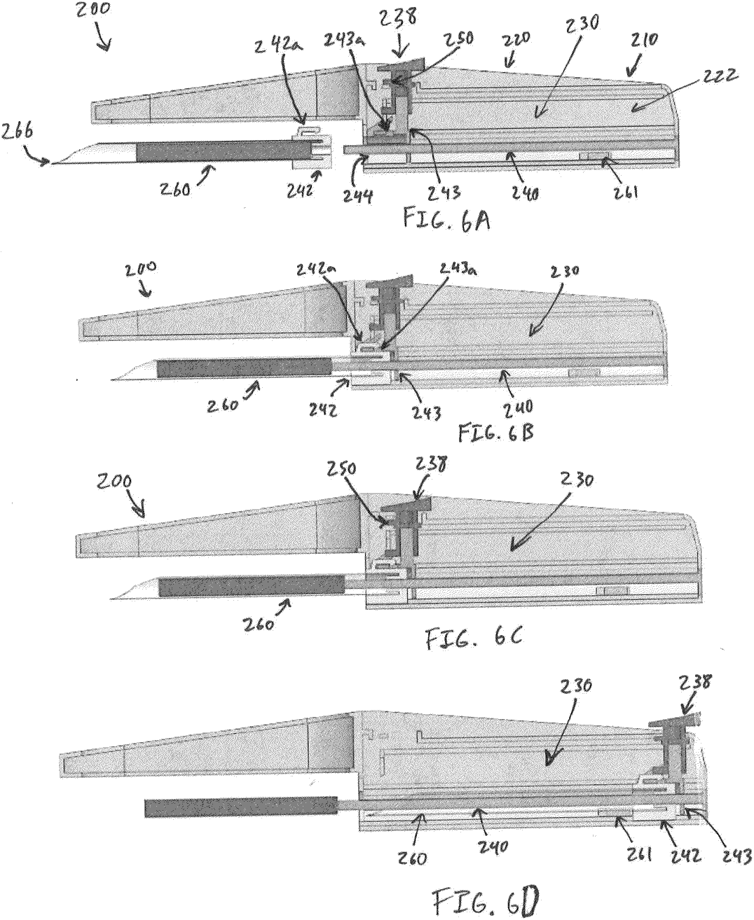

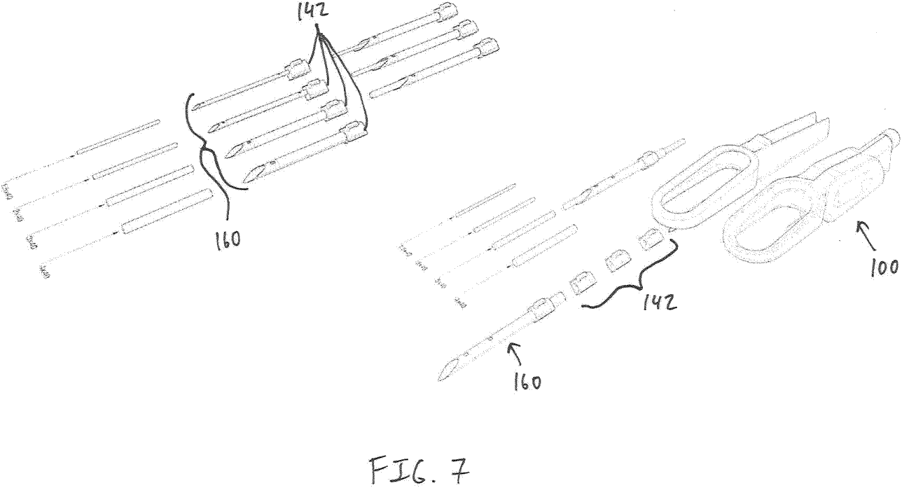

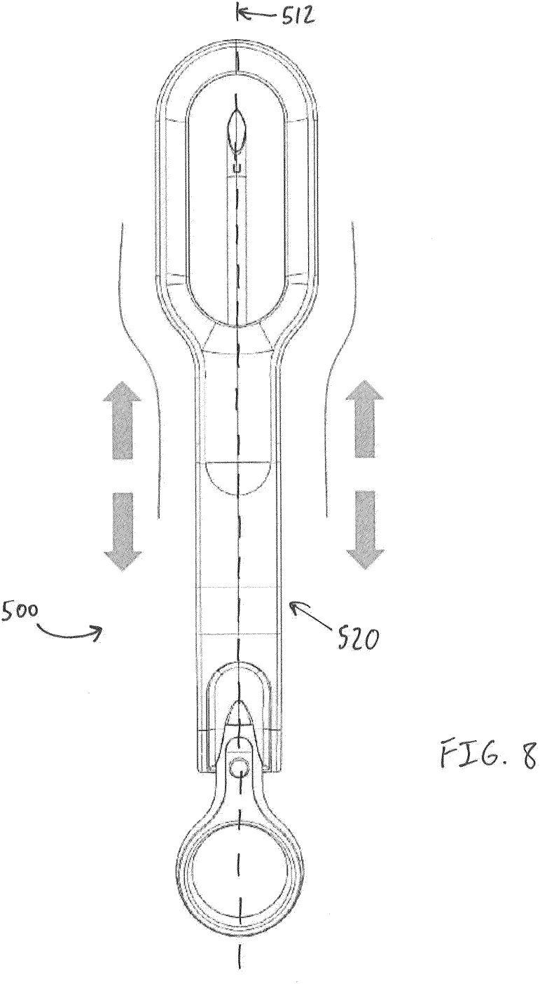

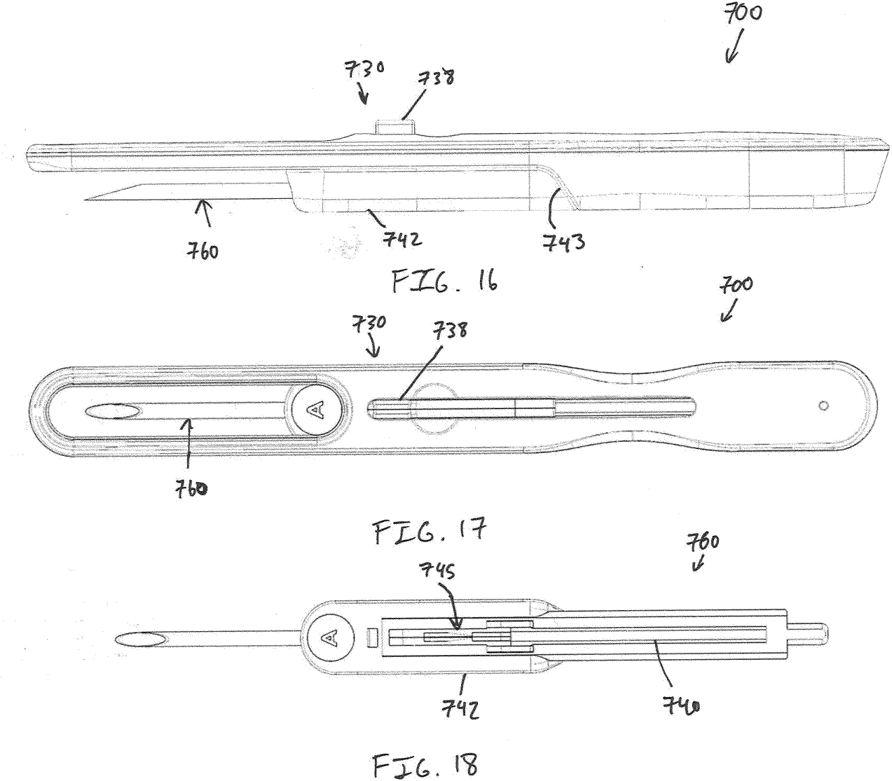

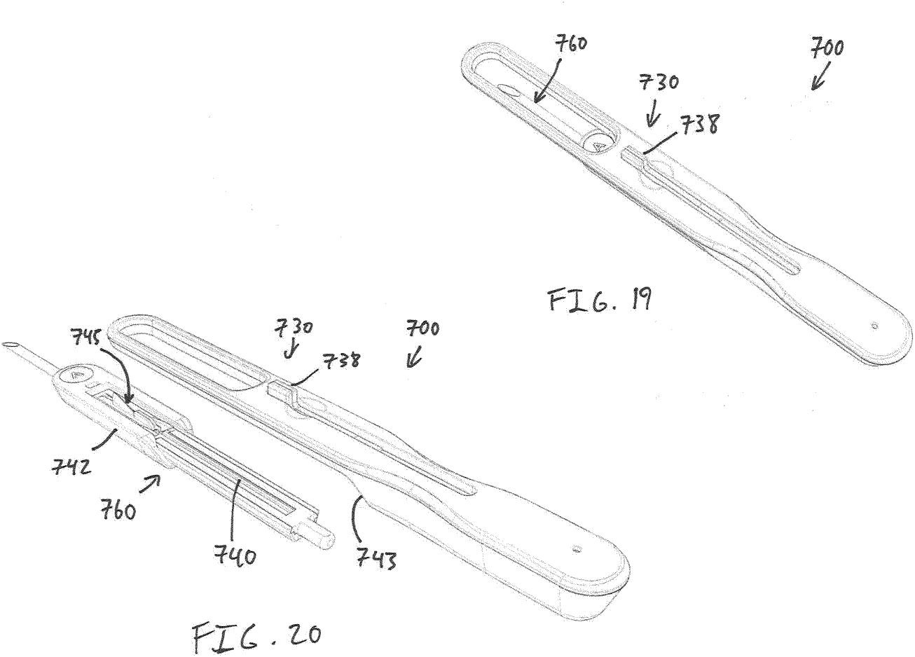

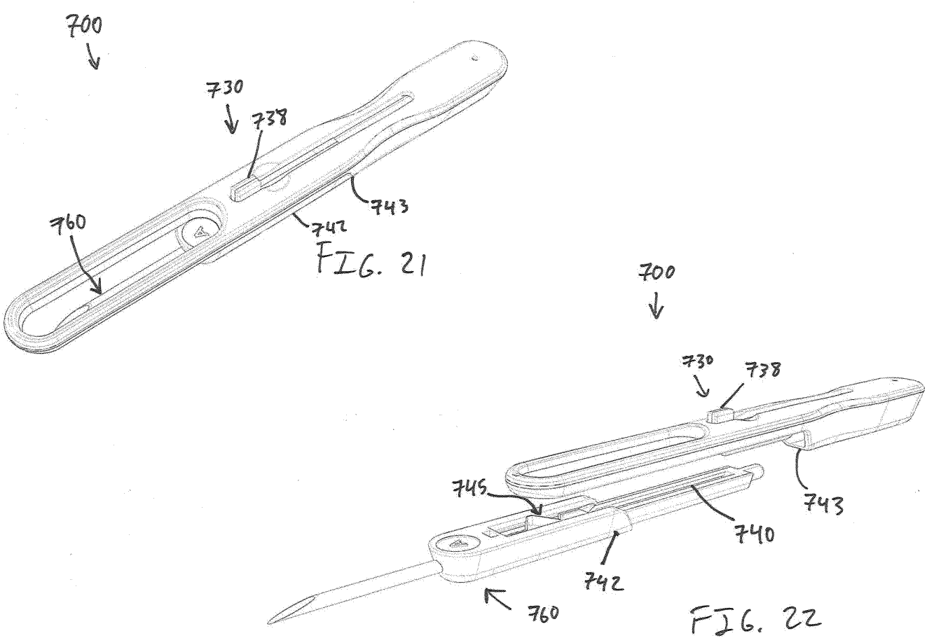

Although the first end 134 of the pull rod 132 shown in FIG. 1-5B includes a pull knob 138, in other implementations, the pull rod includes a handle, a loop, or any other feature that allows the user to grip the first end of the pull rod when exerting a force on the pull rod. In some implementations, such as the device shown in FIGS. 6A-6D and 12-22, the retractor 230, 630, 730 includes a slider 238, 638, 738 directly coupled to the second needle coupling portion 243, 643, 743 such that sliding of the slider 238, 638, 738 causes retraction of the second needle coupling portion 243, 643, 743 and the needle 260, 660, 760. In some implementations, the first end of the pull rod does not include a pull knob or any other feature.

In some implementations, such as the device 700 shown in FIGS. 16-22, the needle further includes the retractor 730 which has a slide tab 745 and a plunger 740. The slide tab 745 is coupled to the needle 760, and the needle 760 is slidable along the plunger 740, similar to the retractors described above. The first needle coupling portion 742 is a body that is removably couplable to the second needle coupling portion 743. When the first needle coupling portion 742 is coupled to the second needle coupling portion 743, the slide tab 745 engages a slider 738 such that movement of the slider 738 cause the movement of the slide tab 745 and needle 760 from the extended position to the retracted position.

In some implementations, the retractor includes a spring to exert a force in a direction away from the handle body to cause the needle coupling portion to move from the extended position to the retracted position. The retractor further includes a trigger that holds the spring in tension until a user actuates the trigger to release the spring force to move the needle coupling portion.

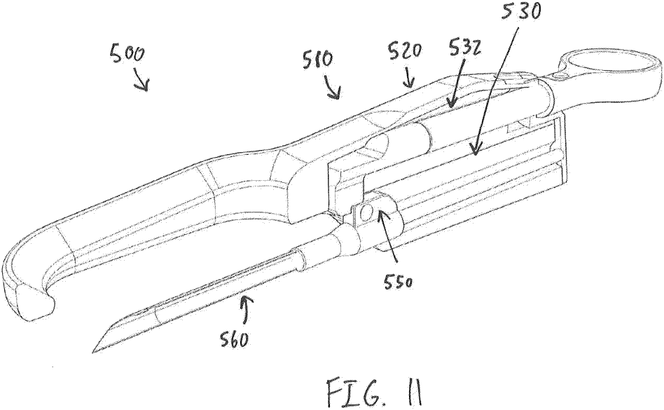

In some implementations, such as the implementation shown in FIG. 11, the retractor 530 further includes a lock 550 that prevents the needle 560 from being inadvertently retracted into the handle body 520 when coupling the needle 560 to the handle 510 or when positioning the needle 560 for insertion into the skin. The pull rod 532 of the retractor 530 of the device 500 can be retracted to break the lock 550 to allow the needle 560 to retract into the handle body 520.

The needle coupling portions 142, 143 each include rotational clocking features that, when the needle coupling portions 142, 143 are coupled to each other, interface to ensure that the distal tip 166 of the needle 160 is rotationally oriented correctly such that the proper entry angle is achieved.

The needle coupling portions 142, 143 shown in FIG. 1-5B includes a luer lock connector for coupling needles 160 that have a mating luer lock connector, but in other implementations, the needle coupling portion can include various snap connections, bayonet connections, or any other type of connector capable of coupling a needle to the retractor. For example, the implementation shown in FIGS. 6A-6D includes a first needle coupling portion 242 and a second needle coupling portion 243. The first needle coupling portion 242 includes a latching arm 242a, and the second needle coupling portion 243 defines a receiver opening 243a configured for receiving a portion of the latching arm 242a. The first needle coupling portion 242 is insertable into a portion of the opening 244 of the second needle coupling portion 243 such that a portion of the latching arm 242a couples to the receiver opening 243a. The coupling of the portion of the latching arm 242a to the receiver opening 243a ensures that the distal tip 266 of the needle 260 is rotationally oriented correctly such that the proper entry angle is achieved.

The needle 160 shown in FIG. 1-5B is a trocar needle. The needle 160 has a longitudinal axis 162, a connection end 164, and a distal tip 166 opposite and spaced apart from the connection end 164 along the longitudinal axis 162. As seen in FIG. 1-5B, the first needle coupling portion 142 of the needle 160 is couplable to the second needle coupling portion 143 of the handle 110 such that the plunger 140 is aligned with the longitudinal axis 162 of the needle 160 and the distal tip 166 of the needle 160 is disposed along the central plane 112.

Although the needle 160 shown in FIG. 1-5B includes a first needle coupling portion 142 attached to the needle 160, in other implementations, such as the needle 460 shown in FIG. 10, the needle includes an integrated first needle coupling portion defined by the needle 460 itself. The connection end 464 defines two openings 465 for engaging with and coupling to a second needle coupling portion. The connection end 464 of the needle 460 also defines a groove 463 for engaging a protrusion of the second needle coupling portion to rotationally clock the needle coupling portions relative to each other to ensure that the distal tip 466 of the needle 460 is rotationally oriented correctly such that the proper entry angle is achieved.

The needle 160 shown in FIG. 1-5B is preloaded with one or more pellets of a drug disposed within the needle lumen prior to the needle 160 being coupled to the needle coupling portion 142 of the retractor 130 of the handle 110. However, in some implementations, the needle is preloaded with rods, cylinders, spheres, capsules, or any other size or shape solid media for subdermal insertion, and the needle is sized for the size and shape of the drug.

In some implementations, the pellets, rods, cylinders, spheres, capsules, containers, or other insertion component are not a drug, but are a device or an implant such as trackers, sensors, microchips, or other type of digital media.

As shown in FIG. 7, in some implementations, the device is configured to be coupled to needles with various lengths and diameters. Even though the diameters and lengths of the needles shown in FIG. 7 vary, the first needle coupling portion is the same size such that the first needle coupling portion of any length or diameter needle is couplable to a generic sized second needle coupling portion. The different lengths and diameters of the needles allow for the same device to be usable with different sizes of solid insertable media.

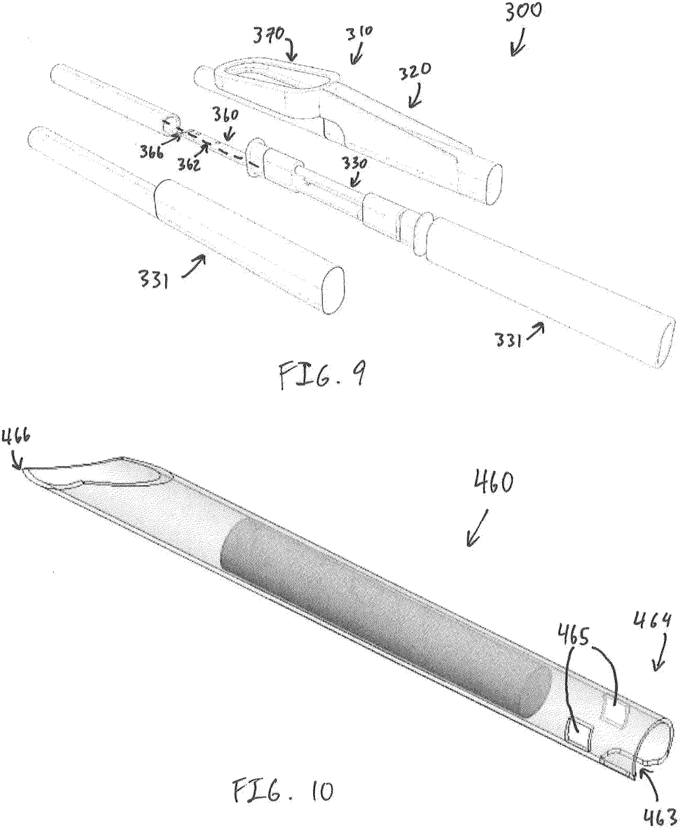

FIG. 9 shows a device 300 according to another implementation. The device 300 is similar to other devices disclosed herein, but the device 300 shown in FIG. 9 includes a preloaded needle cassette 331 that includes a retractor 330 and a needle 360 permanently coupled to the retractor 330. The cassette is insertable into and couplable to a handle body 320 of a handle 310. The size and shape of the cassette 331 is a universal size such that cassettes 331 having various sized needles 360 can be used with a generic handle 310. A portion of the cassette has an ovate cross-sectional shape as viewed in a plane perpendicular to the longitudinal axis 362 of the needle 360 such that the distal tip 366 of the needle 360 is rotationally clocked to a correct position relative to the guide 370 to ensure a proper entry angle. Because the cassette includes all of the internal elements, the internal elements of the cassette remain sterile prior to and during the entire workflow. The internal elements of the cassette can also be optimally sized for the solid media payload included in the needle of the cassette.

In some implementations, such as the device 600 shown in FIGS. 12-15, the needle is permanently coupled to the needle coupling portion of the handle and may be preloaded with the solid media. Thus, in these implementations, a point of care user would not need to couple a needle to the handle prior to use.

The guide 170 is coupled to the handle 110 and extends along a longitudinal axis 172 that is parallel to the needle longitudinal axis 162. The guide 170 has a proximal end 174 coupled to the handle 110 and a distal end 176 opposite and spaced apart from the proximal end 174. The distal end 174 of the guide 170 is disposed along the central plane 112.

The distal end 176 of the guide 170 is disposed further than the distal tip 166 of the needle 160 from the handle 110 when the needle 160 is in the extended position. The length of the guide 170 and the length of the needle 160 shown in FIG. 1-5B are sized such that an axis 178 including the distal end 176 of the guide 170 and the distal tip 166 of the needle 160 forms an entry angle 180 of 30 degrees with the needle longitudinal axis 162. Thus, when the device 100 is used, the user can determine the desired 30 degree angle of entry of the needle 160 by placing the distal end 176 of the guide 170 and the distal tip 166 of the needle 160 on the skin of the patient. Although the entry angle 180 of the device 100 shown in FIG. 1-5B is 30 degrees, in other implementations, the entry angle is any angle in the range of 5-50 degrees, such as 20-40 degrees. In some implementations, the entry angle is any angle in the range of 5-15 degrees. In some implementations, the entry angle is any angle of 15 degrees or less. In some implementations, the entry angle is any angle of 10 degrees or less. The first and second needle coupling portions 142, 143 ensure the correct needle 160 length relative to the guide 170 to establish the desired entry angle 180 while also creating a secure and stable coupling of the needle 160 to the handle 110 that minimizes angular deflection along the cantilevered needle 160.

The guide 170 has a first surface 182 and a second surface 184 opposite the first surface 182. The second surface 184 of the guide 170 is closer than the first surface 182 to the needle 160. The guide 170 shown in FIG. 1-5B further defines a window 190 extending from the first surface 182 to the second surface 184 through which the distal tip 166 of the needle 160 is visible when viewing the needle 160 along the central axis 112 from the first surface 182 toward the second surface 184. Thus, a user can view the distal tip 166 of the needle 160 from the top of the device 100 and through the guide 170 while inserting the needle 160 into a patient.

Although the guide 170 of the device 100 shown in FIG. 1-5B defines a window 190, it should be appreciated that, in other implementations, the guide can have any shape that allows the distal tip of the needle to be viewed along the central plane and perpendicular to the axis including the distal end of the guide and the distal tip of the needle and still includes a distal end of the guide that can be used to establish an entry angle, such as a forked guide or a curved guide. In some implementations, the guide is made of a transparent or translucent material such that the distal tip of the needle can be viewed through the guide.

The edge where the second surface 184 and the distal end 176 of the guide 170 shown in FIG. 1-5B intersect defines a fillet 186 for guiding the angle of the needle 160 during insertion. However, in other implementations, the edge of the second surface and the distal end of the guide includes a chamfer, a bevel, or any other eased edge capable of guiding the angle of the needle during insertion.

The second surface 184 of the guide 170 defines a plane that extends parallel to the needle longitudinal axis 162 and has a total width 192 of 15 mm as measured across the entire second surface 184. The width 192 of the second surface 184 of the guide 170 provides stability of the device 100 during insertion of the needle 160 to prevent undesired rotation of the device 100 during use. Although the total width 192 of the second surface 184 of the device 100 shown in FIG. 1-5B is 15 mm, in some implementations, the width of the second surface is in the range of 15 mm-30 mm. In some implementations, the width of the second surface is 15 mm or larger. In some implementations, the width of the second surface is three-five times larger than the outside diameter of the needle. In some implementations, the width of the second surface is three or more times larger than the outside diameter of the needle.

The distance 194 from the second surface 184 of the guide 170 to the needle 160 of the device 100 shown in FIG. 1-5B is 3 mm-15 mm, preferably 5 mm-10 mm, depending on the typical thickness of the skin in the intended body location. However, in other implementations, the distance from the second surface of the guide and the needle is any distance chosen based on a desired depth of insertion of the needle.

Referring to FIGS. 6A-6D, in use, a user slides a needle 260 into the opening 244 such that the first needle coupling portion 242 of the needle 260 couples with the second needle coupling portion 243 of the retractor 230, as shown in FIGS. 6A and 6B. The user abuts the distal tip 266 of the needle 260 and the distal end 276 of the guide 270 against the skin of a patient to establish an entry angle 280. The user then exerts a force on the handle 210 of the device 200 to cause the needle 260 to penetrate the skin. By keeping the guide 270 contacted with the skin of the patient, the angle of insertion of the needle 260 is rotated about the fillet 286 on the edge of the guide 270, limiting the depth of insertion of the needle 260 into the patient. Once the device 200 is rotated such that the second surface 284 of the guide 270 is contacting the skin of the patient and the needle 260 is generally parallel to the surface of the skin, the needle 260 is fully inserted under the skin. The user then activates the lock 250, for example, by exerting enough force on a slider of the retractor 230 to depress the slider past the lock 250 such that the slider and the second needle coupling portion are retractable into the handle 210, as shown in FIG. 6C. The retractor 230 is then actuated, either manually by sliding the slider 238, pulling the pull knob, or automatically by actuating the trigger, to move the needle 260 from the extended position to the retracted position, as shown in FIG. 6D. When the needle 260 is moved from the extended position to the retracted position, the needle 260 is removed from under the skin of the patient and into the cavity 222 of the handle body 220 such that the distal tip 266 of the needle 260 is no longer exposed. The device can also include a second lock 261, such as the barb-type lock shown in FIGS. 6A-6D to prevent the needle 260 from moving from the retracted position back toward the extended position.

The plunger 240 of the retractor 230 is statically coupled to the handle body 220 and is aligned with the needle 260 such that, as the needle 260 is moved from the extended position to the retracted position, the plunger 240 extends through the lumen of the needle 260, as shown in FIG. 6D. Because the plunger 240 remains static with respect to the handle body 220 when the needle 260 is retracted, the solid media disposed within the lumen of the needle 260 contact the end of the plunger 240 and are prevented from retracting with the needle 260 such that the solid media is left under the skin of the patient. With the needle 260 retracted into the cavity 222 of the handle body 220, no portion of the device 200 must be further withdrawn from the patent.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

1. A device for subdermal insertion of solid media, the device comprising:
a handle having a central plane;
a needle coupled to and extending from the handle, the needle having a needle longitudinal axis and a distal tip disposed along the needle longitudinal axis and the central plane; and
a guide extending from the handle, the guide having a distal end disposed along the central plane,
wherein the distal end of the guide is disposed further than the distal tip of the needle from the handle, and
wherein the distal tip of the needle is visible when viewed along the central plane and perpendicular to an axis including the distal end of the guide and the distal tip of the needle.

2. The device of claim 1, wherein an axis including the distal end of the guide and the distal tip of the needle forms an entry angle with the needle longitudinal axis, the entry angle being in the range of 5-50 degrees.

3. (canceled)

4. The device of claim 2 , wherein the entry angle is in the range of 5-15 degrees.

5. The device of claim 1, wherein the guide defines a window through which the distal tip of the needle is visible.

6. The device of claim 1, wherein the handle defines a cavity and the needle is retractable into the handle.

7. The device of claim 6, wherein the needle is manually retractable.

8. (canceled)

9. The device of claim 6, wherein the handle includes a slider for causing the needle to move from an extended position to a retracted position.

10. The device of claim 6, further including a plunger axially aligned with the needle longitudinal axis such that, when the needle is retracted, the plunger extends through the needle.

11. The device of claim 6, wherein the needle is lockable in a retracted position.

12. (canceled)

13. The device of claim 1, wherein the handle has an hourglass shape as viewed in a direction parallel to the central plane.

14. The device of claim 1, wherein an edge of the distal end of the guide closest to the distal tip of the needle defines a fillet, a chamfer, or an eased edge.

15. The device of claim 1, wherein the needle includes a first needle coupling portion and the handle includes a second needle coupling portion and the first needle coupling portion of the needle is coupled to the second needle coupling portion of the handle.

16. The device of claim 15, wherein the needle and the handle include rotational clocking features to ensure the rotational position of the needle relative to the handle.

17. The device of claim 15, wherein the first needle coupling portion is integrally formed with the needle.

18. The device of claim 15, wherein the needle is preloaded with one or more pellets, rods, capsules, cylinders, spheres, implants, sensors, or any combination thereof.

19. The device of claim 1, wherein the guide includes at least one surface that defines a plane that extends parallel to the needle longitudinal axis, wherein a total width of the at least one surface is 15 mm-30 mm.

20. The device of claim 1, wherein the guide includes at least one surface that defines a plane that extends parallel to the needle longitudinal axis, wherein a total width of the at least one surface is three or more times larger than an outer diameter of the needle.

21. The device of claim 1, wherein the guide includes a surface that is closest to the needle, wherein a distance from the surface of the guide to the needle is 3 mm-15 mm.

22. The device of claim 21, wherein the distance from the surface of the guide to the needle is 5 mm-10 mm.

23. A device for subdermal insertion of solid media, the device comprising:
a handle having a central plane and a second needle coupling portion for coupling a first needle coupling portion of a needle, the needle having a needle longitudinal axis and a distal tip disposed along the needle longitudinal axis, wherein, when the needle is coupled to the needle coupling portion, the needle extends from the handle such that the distal tip of the needle is disposed along the central plane; and
a guide extending from the handle, the guide having a distal end disposed along the central plane.

24-43. (canceled)
